(19) United States
(12) Patent Application Publication
(10) Pub. No.: US 2026/0345166 A1
(43) Pub. Date: Nov. 5, 2026

(54) LAPAROSCOPIC APPLICATOR WITH FLOW RESTRICTION

(71) Applicants: Baxter International Inc., Deerfield, IL (US); Baxter Healthcare SA, Glattpark (Opfikon) (CH)

(72) Inventors: Laura Giesey, Libertyville, IL (US); Jocelyn Moreno-Hernandez, Joliet, IL (US); Piotr Maniak, Palatine, IL (US); Spencer Scott Paris, Mount Prospect, IL (US)

(21) Appl. No.: 19/665,261

(22) Filed: May 1, 2026

Related U.S. Application Data
(60) Provisional application No. 63/798,329, filed on May 1, 2025.

(51) Int. Cl. A61B 17/12

(52) U.S. Cl. CPC A61B 17/12; A61B 2017/12004

(57) ABSTRACT

An improvement in bellows type hemostatic powder application devices provides for acceptable flexibility and positioning and a reduction in unwanted powder spillage. The improvement comprises placing a flow restrictor tube with a very small internal diameter towards the proximal end of an applicator tube and providing a flow path with a series of expansions and contractions to puff up the powder and provide better placement control and consistent spread and coverage of a target site.

### CROSS-REFERENCE TO RELATED APPLICATIONS

The present application claims the benefit of U.S. Provisional Application No. 63/798,329, entitled LAPAROSCOPIC APPLICATOR WITH FLOW RESTRICTION and filed May 1, 2025, the contents of which are hereby incorporated by reference in their entirety.

### BACKGROUND

Control of bleeding is essential and critical in surgical procedures to minimize blood loss, to reduce post-surgical complications, and to shorten the duration of surgery in the operating room. During laparoscopic surgery minimal invasion incisions are used that help surgeons reduce patient infection and recovery time. However, it is challenging to apply products during the operation as the incisions typically allow for only 5 mm wide ports. During surgery, both expected and unexpected bleeding can occur. Applying a product to control bleeding at the right place and time is critical.

In laparoscopic surgery, patients may have multiple ports with trocars facilitating the insertion of medical devices. For controlling bleeding, a surgeon may utilize a grasper tool through one trocar and employ a second trocar with an applicator to deliver hemostatic powder from the bellows to the bleeding site. To administer the hemostatic powder, the surgeon introduces the applicator into the trocar and manipulates the handle or graspers to bend, twist, and rotate the applicator's free end towards and in proximity to the bleeding site. Conventional applicators may cause hemostatic powder to inadvertently leak during movement of the applicator and handle with the bellows container, potentially resulting in less precise delivery at the target location.

Various approaches to addressing the challenge of powder loss were evaluated. The implemented solution is required to make minimal modifications to the existing manufacturing process. The resultant applicator should demonstrate a significant degree of flexibility while controlling powder flow.

Certain proposed solutions included crimping the applicator tube; however, this was incompatible with the manufacturing process and could reduce the tube's size excessively. Another proposal involved integrating a flow restriction within the handle, which necessitated a mold modification, thereby incurring increased costs and not aligning with the requisite timeline. An additional approach entailed adding extra crimping at the handle-tube junction, but this posed inconsistencies with the manufacturing process and introduced risks of handle damage. Another suggestion was to apply an external clamp to the applicator tube; however, this reduced the tube length available to the user. Furthermore, an attempt to design the applicator tube with a smaller inner diameter at the handle end and a larger diameter towards the distal end presented manufacturing challenges within the required timeframe. An applicator that demonstrates substantial flexibility while managing powder flow remains a necessity.

### SUMMARY

The present device provides an improved hemostatic powder laparoscopic applicator with good flexibility and improved powder placement control.

The present device comprises an improvement in a bellows type hemostatic powder laparoscopic applicator assembly of the type. Example systems, methods, and apparatuses are disclosed herein for a hemostatic powder laparoscopic applicator with good flexibility and improved powder placement control. The systems, methods, and apparatuses are configured to apply a hemostatic powder to control bleeding, to minimize blood loss during surgery, to reduce post-surgical complications, and shorten the duration of surgery. The applicator described herein has a high degree of flexibility while restricting powder flow

In light of the disclosure herein and without limiting the disclosure in any way, in a first aspect of the present disclosure, which may be combined with any other aspect listed herein, a hemostatic powder laparoscopic applicator device comprises an applicator tube, which includes a proximal applicator end and a distal applicator end.

The distal applicator end comprises a powder discharge opening. The device also includes a flow restriction tube positioned at the proximal applicator end, wherein the flow restriction tube comprises a proximal tube end and a distal tube end. Further, the device includes a handle member comprising a proximal handle end and a distal handle end, wherein the proximal handle end includes a proximal handle end opening. Moreover, the device includes a first crimp, a second crimp, and a hollow container comprising a proximal container end and a distal container end. The distal container end includes a distal powder opening. The hemostatic powder laparoscopic applicator comprises an inner diameter allowing the hemostatic powder to flow from the distal container opening to the powder discharge opening. The first crimp is configured to secure the proximal applicator end and the distal handle end, the second crimp is configured to secure the distal tube end to the applicator tube, and the first crimp and the second crimp are configured to modify the inner diameter.

In a second aspect of the present disclosure, which may be combined with any other aspect listed herein, the hemostatic powder applicator includes a first crimp and a second crimp, wherein the first crimp and the second crimp are configured to hold the flow restriction tube in place and to allow flow of powder from a distal container opening to the powder discharge opening.

In a third aspect of the present disclosure, which may be combined with any other aspect listed herein, the inner diameter at the first crimp is larger than the inner diameter at the second crimp.

In a fourth aspect of the present disclosure, which may be combined with any other aspect listed herein, the applicator tube and the flow restriction tube are the same material.

In a fifth aspect of the present disclosure, which may be combined with any other aspect listed herein, the flow restriction tube has a sidewall thickness of from about 2.5 mm to about 3 mm and a length about 22.5 mm to about 28 mm.

In a sixth aspect of the present disclosure, which may be combined with any other aspect listed herein, the flow restriction tube has an outer diameter of about 3 mm to about 4 mm and an inner diameter of about 1 mm to about 1.5 mm, and wherein the applicator tube has an internal diameter of about 3.5 mm to about 4 mm and an outer diameter of about 4.5 mm to about 5.5 mm.

In a seventh aspect of the present disclosure, which may be combined with any other aspect listed herein, the applicator tube and the flow restriction tube provide turbulence to hemostatic powder flowing through the inner diameter by providing larger inner diameter portions after and adjacent the distal tube end, the first crimp and the second crimp, respectively.

In an eighth aspect of the present disclosure, which may be combined with any other aspect listed herein, the hemostatic powder includes PerClot® Absorbable Hemostatic Powder.

In a ninth aspect of the present disclosure, which may be combined with any other aspect listed herein, hemostatic powder laparoscopic delivery system includes an applicator tube including a proximal applicator end and a distal applicator end. The distal applicator end comprises a powder discharge opening. A flow restriction tube is positioned at the proximal applicator end, wherein the flow restriction tube comprises a proximal tube end and a distal tube end. A handle member comprises a proximal handle end and a distal handle end. The proximal handle end includes a proximal handle end opening and the distal handle end comprises a distal handle end opening. A hollow container comprising a proximal container end and a distal container end. The hemostatic powder laparoscopic applicator comprises an inner diameter configured to allow the hemostatic powder to flow from a distal powder opening of the container to the powder discharge opening.

In a tenth aspect of the present disclosure, which may be combined with any other aspect listed herein, the hemostatic powder laparoscopic delivery system further includes a first crimp secures the proximal applicator end and the distal handle end and a second crimp secures the distal tube end to the applicator tube, and wherein the first crimp and the second crimp are configured to cause the inner diameter to decrease from the distal container opening to the powder discharge opening.

In an eleventh aspect of the present disclosure, which may be combined with any other aspect listed herein, the proximal applicator end is telescopically received in the distal handle end opening.

In a twelfth aspect of the present disclosure, which may be combined with any other aspect listed herein, the proximal handle end includes a threaded socket.

In a thirteenth aspect of the present disclosure, which may be combined with any other aspect listed herein, the threaded socket is configured to engage with a threaded distal portion of the hollow container.

In a fourteenth aspect of the present disclosure, which may be combined with any other aspect listed herein, the inner diameter at the first crimp is larger than the inner diameter at the second crimp.

In a fifteenth aspect of the present disclosure, which may be combined with any other aspect listed herein, the applicator tube and the flow restriction tube are the same material.

In a sixteenth aspect of the present disclosure, which may be combined with any other aspect listed herein, hemostatic powder laparoscopic delivery system further includes PerClot® Absorbable Hemostatic Powder as the hemostatic powder.

In a seventeenth aspect of the present disclosure, which may be combined with any other aspect listed herein, the hemostatic powder laparoscopic delivery system further includes a finger grip secured to the handle member and comprising two gripping handles.

In an eighteenth aspect of the present disclosure, which may be combined with any other aspect listed herein, the hemostatic powder laparoscopic delivery system further includes a flow restriction tube having an outer diameter of about 3 mm to about 4 mm and an inner diameter of about 1 mm to about 1.5 mm. Further, the applicator tube has an internal diameter of about 3.5 mm to about 4 mm and an outer diameter of about 4.5 mm to about 5.5 mm.

In a nineteenth aspect of the present disclosure, which may be combined with any other aspect listed herein, a method of manufacturing a bellows type hemostatic powder dispenser comprises a container, a handle member, and an applicator tube, the method includes the following steps. Providing a flow restriction tube, inserting the flow restriction tube into the applicator tube to a specific depth within the applicator tube, connecting the handle member to the same side of the applicator tube as the flow restriction tube, inserting the proximal end of the flow restrictor tube into a distal end of the handle member to a specified depth, crimping the handle member, applicator tube and flow restrictor tube at a first location to provide a first crimp, and crimping the applicator tube at a second location distal to the distal end of the flow restrictor tube to provide a second crimp.

In a twentieth aspect of the present disclosure, which may be combined with any other aspect listed herein, the method of manufacturing a bellows type hemostatic powder dispenser further comprises crimping the handle member and crimping the applicator are performed at the same time.

Additional features and advantages are described in, and will be apparent from, the following Detailed Description and the Figures. The features and advantages described herein are not all-inclusive and, in particular, many additional features and advantages will be apparent to one of ordinary skill in the art in view of the figures and description. Also, any particular embodiment does not have to have all of the advantages listed herein and it is expressly contemplated to claim individual advantageous embodiments separately. Moreover, it should be noted that the language used in the specification has been selected principally for readability and instructional purposes, and not to limit the scope of the inventive subject matter.

### BRIEF DESCRIPTION OF THE DRAWINGS

FIG. 1 is a perspective view of the PerClot® Absorbent Hemostatic Powder Applicator with a PerClot® 20 cm Extender Tip in accordance with the prior art.

FIG. 2 is an elevated side view of the PerClot® 38 cm Laparoscopic Tip in accordance with the prior art.

FIG. 3 is an elevated side view of the new and improved laparoscopic applicator in accordance with the present device.

FIG. 4 is an elevated side view of the new and improved laparoscopic applicator of the present device shown partially in section.

FIG. 5 is an elevated cross-sectional view of the new and improved laparoscopic applicator taken along view lines A-A in FIG. 4.

FIG. 6 is an elevated side view of an applicator tube for use in the present device.

FIG. 7 is an elevated cross-sectional view of the applicator tube taken along view lines A-A in FIG. 6.

FIG. 8 is an elevated side view of the flow restrictor tube for use in the new and improved laparoscopic applicator in accordance with the present device.

FIG. 9 is an elevated cross-sectional view of the flow restrictor tube taken along view lines A-A in FIG. 8.

FIG. 10 is an elevated cross-sectional view of the first crimp section in the new and improved laparoscopic applicator.

FIG. 11 is an elevated cross-sectional view of the second crimp section in the new and improved laparoscopic applicator.

FIG. 12 is a schematic view of a practice apparatus for laparoscopic surgery shown including two ports with trocars, one having a gripper tool inserted into a simulated body cavity and one having a PerClot® 38 cm Laparoscopic Tip in accordance with the prior art to illustrate the problem solved by the present device.

### DETAILED DESCRIPTION

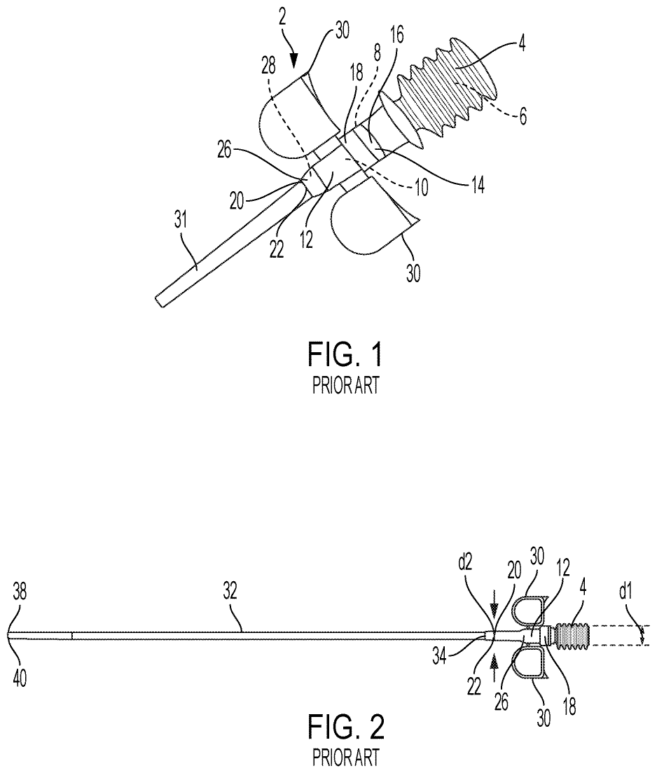

A PerClot® Absorbable Hemostatic Powder Device is shown in FIGS. 1-2. More particularly, the PerClot® Absorbable Hemostatic Powder applicator in FIG. 1 is shown with a PerClot® 9 cm Standard Tip handle connected to the bellows container. The hemostatic powder dispenser assembly 2 shown in FIG. 2 depicts the PerClot® Powder device connected to a PerClot® 38 cm Laparoscopic Tip. This design is directed to the laparoscopic version shown in FIG. 2.

FIG. 1 discloses a conventional hemostatic powder dispenser assembly 2 that includes a hollow bellows container 4, a handle member 12 and an applicator extender 31, which are all formed from one molded plastic part. In FIG. 2, the hollow bellows container 4 is located at a proximal end of the assembly 30, the applicator tube 32 is located at a distal end of the assembly 30, and a handle member 12 is located at a distance from the proximal end and at a distance from the distal end of the assembly 2. In FIG. 2, the handle member 12 is separate from the applicator tube 32. The applicator tube 32 is fitted to the handle member tube by mechanically crimping. Specifically, a pin (not disclosed) is pushed through handle member 12 into the applicator tube 32 to lock them together. The mechanically crimped applicator tube 32 and handle member 12 is a sellable unit.

Hollow bellows container 4 is configured to be loaded with an amount of a hemostatic powder 6, which is illustrated in broken lines. The hollow bellows container 4 includes a distal end with a threaded distal portion 8 with a distal powder opening 32. The proximal end of the hollow bellows container is closed. The hollow bellows container 4 does include a threaded cap (not shown) which is removed prior to connecting the bellows container into the handle socket 18. To connect the hollow bellows container 4 with the hemostatic powder, a user will detach the cap of the hollow bellows container 4 from the hemostatic powder dispenser and screw the bellow container 4 onto the assembly 2 or assembly 30 . . . . The hollow bellows container 4 is provided separately from the handle member 12 and the applicator tube 32 and the hollow bellows container 4 is pre-filled with the hemostatic powder.

In the device disclosed in FIG. 2, the diameter of the hollow bellows container 4 does not include any variations configured to restrict and manipulate the flow of hemostatic powder. A user merely compresses and expands the hollow bellows container 4 to dispense the hemostatic powder 6 using the assembly 2. For example, the configuration of the hollow bellows chamber 4 allows a user to apply force to the proximal end of the hollow bellows container, creating pressure within the chamber 4, and propelling the powder through the connected handle 12 or assembly 30. Thus, the hollow bellows container 4 is configured to accommodate a predetermined amount of hemostatic powder 6. However, this method of application (e.g., merely applying force to the hollow bellows 4) does not allow for a user to control the amount of hemostatic powder delivered from a laparoscopic applicator to ensure precise application at the bleeding site. Controlled delivery helps maintain clear visualization during surgery and prevents excess powder from dispersing to surrounding tissues. It also reduces the risk of complications from over-application and ensures efficient use of the hemostatic agent.

Thus, there exists a need to develop a new and improved laparoscopic applicator that is configured to improve the control of the amount of hemostatic powder leaving the device.

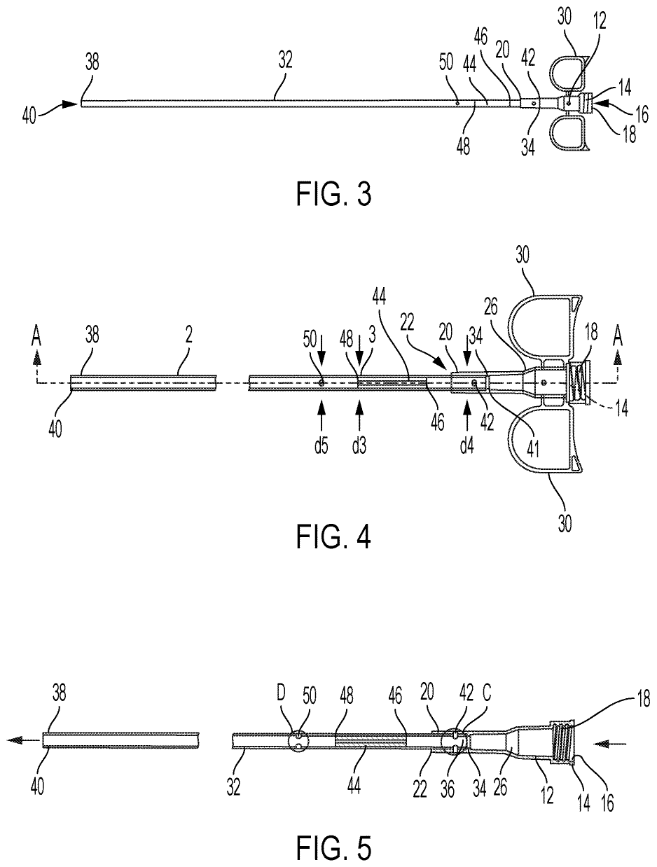

FIGS. 3-5 illustrate a new and improved laparoscopic applicator including a handle member 12 attached to an applicator tube 32, which has been found to result in improved control during the application of hemostatic powder. The handle member 12 includes a proximal handle end 14 and a distal handle end 20. A first inner diameter d1 is defined at the proximal handle end 14 and a second inner diameter d2 is defined in the distal handle end 20. In some embodiments, d1 is greater than d2. D1 is from about 1.3 cm to about 1.7 cm. D2 is about from 0.6 cm to about 0.8 cm.

The proximal handle end 14 of the handle 12 includes a proximal handle end opening 16. The proximal handle end 14 includes a threaded socket 18. The threaded socket 18 may include outer threads, which are configured to engage with a threaded distal portion 8 of the hollow bellows container 4 (not disclosed in FIGS. 3-5), ensuring a stable and airtight engagement. This arrangement minimizes the risk of powder leakage and ensures efficient transfer of hemostatic powder into the applicator tube during usage.

The distal handle end 20 of handle 12 includes a distal end opening 22. The body of the handle (e.g., the portion of the handle between the proximal handle end 14 and the distal handle end 20) is the middle section 26. The middle section 26 can have varying diameters. For example, the middle section 26 is frustoconical. In an example embodiment, the middle section 26 includes a larger diameter at a first portion of the middle section 26 and a smaller diameter at a section portion of the middle section 26. The larger diameter is the diameter closest to the proximal handle end 14 and the smaller diameter is the diameter closest to the distal handle end 20.

The handle 12 includes an inner powder passageway 29 that extends from the distal end 12 to the proximal handle end 14. The inner powder passageway is a hollow portion inside the handle 12 allowing powder to flow from the container 4, through the handle 12, and into the applicator tube 32. A diameter of the inner powder passageway may be adjusted based on a desired level of flow of powder. For example, However, it is preferred for the diameter of the inner powder passageway to be tapered. A tapered inner powder passageway may improve flow consistency, reduces clogging, and enhances metering control.

FIGS. 3-4 also provide an example embodiment of a handle 12 including a grip 30. The grip 30 projects outwardly from the handle 12 such that a user may hold the device with, for example, one or two fingers. The grip 30 may be configured such that a user can merely hold the handle 12 portion of the assembly or may be configured such that a user may wrap at least one finger around the grip 30.

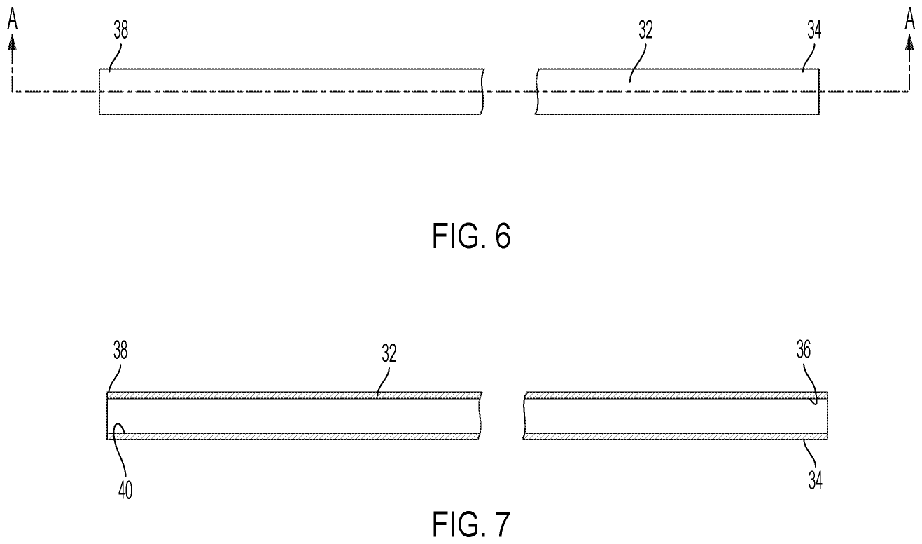

The hemostatic powder dispenser assembly 12 further includes the elongate applicator tube 32, as illustrated in FIGS. 2-5, 6-7 and 10-11. The applicator tube 32 has a proximal applicator end 34 with an opening 36 and a distal applicator end 38. The distal applicator end includes a powder discharge opening 40. In an illustrative embodiment, the proximal applicator end 34 of applicator tube 32 may be fixed in place via mechanical crimping, which is described later.

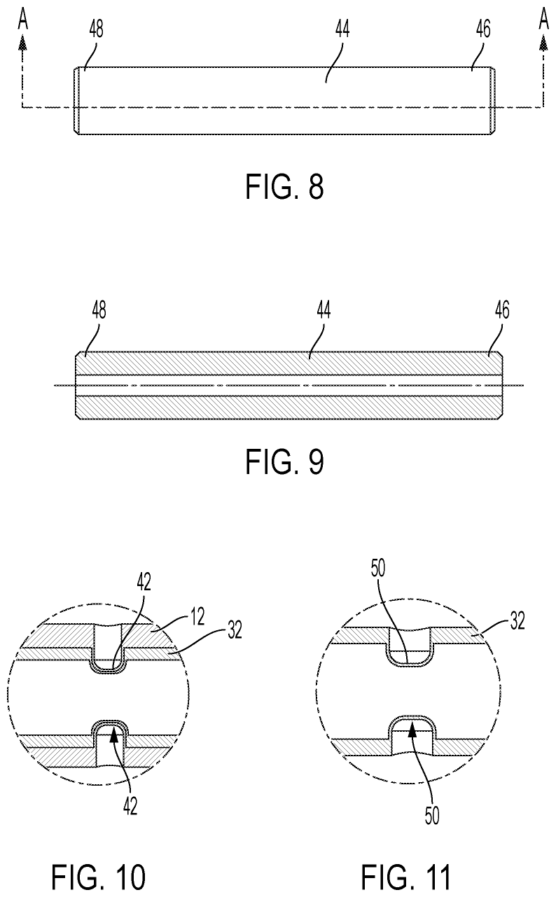

As shown in FIGS. 4-5 and 8-11, an inner flow restriction tube member 44 is located in a lumen of the applicator tube 32. The inner flow restriction tube member 44 is located at a distance from the distal applicator end 38 and at a distance from the proximal applicator end 34, wherein the distance from the proximal applicator end 34 is preferably smaller than the distance from the distal applicator end 38. In other words, the inner flow restriction tube member 44 is located toward the proximal applicator end 34. However, it is foreseeable for the inner flow restriction tube member 44 to be located anywhere along a length of the lumen of the applicator tube 32 or can be located in the adapter of the bellows or anywhere else in the handle. Flow restriction tube 44 has a proximal flow restriction end 46, a distal flow restriction end 48 and a central flow path provided by inner diameter d3. Preferably, inner diameter d3 is from about 1.04 mm to 1.24 mm, more preferably about 1.14 mm.

As is best shown in FIGS. 5, 10 and 11, the flow restriction tube member 44 is held in place within the applicator tube 32 in between a first crimp 42 and a second crimp 50. First crimp 42 is configured to connect applicator tube 32 and handle member 12 by crimping or applying force to a circumferential indent in the sidewalls of applicator tube 32 and handle member 12. The first crimp holds the handle member 12 and the applicator tube 32 together and force applied will reduce the diameter of the flow path to a smaller inner diameter, d4. This crimp can be included or excluded from the design as long as the handle is attached to the cannula by other means such as ultrasonic welding. The handle and the cannula are sold to the customer as a one piece product to apply the powder.

In accordance with this device, the second crimp 50 is located in the applicator tube 32 distal to and spaced from the distal end 48 of flow restrictor tube 44. The second crimp 50 is made by crimping a circumferential indent in applicator tube 32 defining another narrowed inner diameter, d5, to the flow path in the applicator tube 32. The first crimp 42 and the second crimp 50 also maintain the flow restrictor tube 44 in place between the first crimp 42 and the second crimp 50 such that the inner flow restriction tube member 44 does not move beyond the second crimp 50 towards the distal applicator end 38 and risk fall out of the applicator tube 32.

In a preferred embodiment, the inner flow restriction tube member 44 is friction fit within the applicator tube 32 and further is located closer to the proximal applicator end 34 than the distal applicator end 38.

In the example embodiment of FIG. 5, which is disclosed with a broken applicator tube 32 for illustrative purposed only, the flow path moving from right to left begins with the distal powder opening 10 of the hollow bellows container 4 and the mated complimentary proximal handle end opening 16 in threaded socket 18 and an inner diameter (ID) d1. The flow path continues within the handle member 12 through the middle section 26 to the distal handle end 20 with its distal end opening 22 and the second ID d2. This ID d2 continues into the proximal applicator end 34 of applicator tube 32, ID d3 up to the first crimp 42 with its ID d4. After first crimp 42, the ID of the powder path enlarges to the ID d3 of the applicator tube 32 until the proximal flow restriction end 46 opening of flow restrictor tube 44 is reached. The ID of flow restrictor tube narrows the passageway and flow path to ID d5 until the distal flow restriction end 48 of the flow restrictor tube 44 is reached. Thereafter, the powder path enlarges again to the ID d3 of the applicator tube until the second crimp 50. The second crimp 50 narrows to ID d6. After the second crimp 50 the ID enlarges again to the ID d3 for the remainder of the applicator tube 32.

The inventors have discovered that the configuration of the diameter of the flow restrictor tube 44 advantageously provides the ability to puff up the powder with turbulence as it passes through the flow path such that the discharged powder from powder discharge opening 40 is consistent, full and provides good coverage for the target bleeding site.

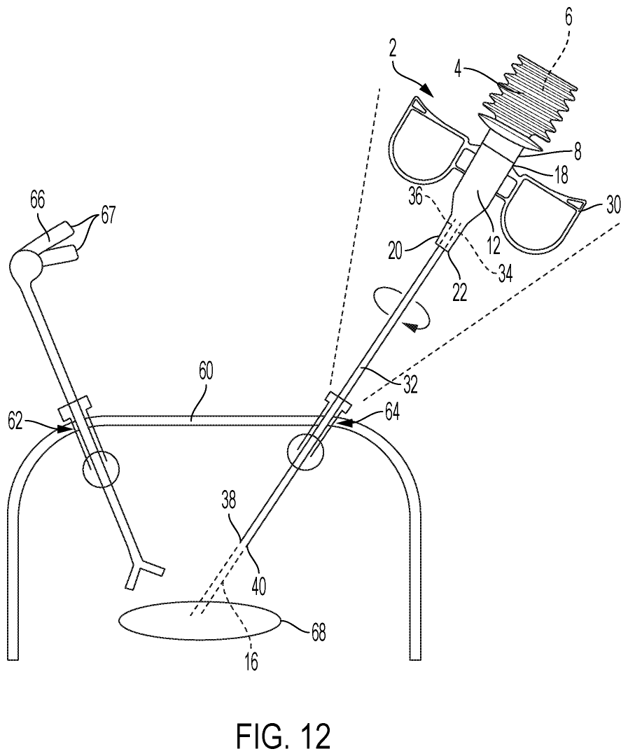

FIG. 12 provides an example embodiment of a schematic view of a clear dome 60. Clear dome 60 defines a clear chamber, which is intended to represent a human body. Two trocar ports 62, 64 are provided. An elongate surgical gripper instrument 66 is extended through the trocar 62 into clear chamber 60 which may represent an abdominal cavity. The grasper instrument 66 has finger actuators 67 which remain on the outside of dome 60 for controlling the gripper jaws. Trocar port 64 includes a hemostatic powder delivery device 2. A target area 68 of bleeding is illustrated.

In an illustrative example, a user, such as a surgeon or clinician, may manipulate the distal end of a laparoscopic applicator tube 32 into a position in close proximity to the target. In doing so, the bellows 4 are connected to the handle 12 and the applicator tube 32 is inserted into the dome 60 to the inner cavity. When the handle 12 is inverted above the trocar, often with prior devices, the hemostatic powder simply spills out of the bellows container all the way through the handle and applicator tube. This unintended accidental spillage is a shortcoming because it does not get the hemostatic powder to the right place at the right time. Bleeding times will remain longer and the surgeon may need to open another hemostatic powder if there is not enough left in the bellows 4. In some cases, all of the hemostatic powder comes out unintentionally which creates waste.

Referring again to FIG. 12, operating under stressful conditions, sudden and unexpected bleeding shows up in various kinds of locations. The surgeon is motivated to get there fast and in the process, bends and pulls on the applicator 32 and bellows 4 container to the site of bleeding. During these actions the applicator tube 32, and handle 12, and bellows 4 may pass through vertical many times with shaking movements, which make it easy to understand the way the problem of spillage arises if there in not flow restriction provided.

The inventors have experimented with a variety of applicator tubes 32 with smaller IDs, such as 3.0 mm, 2.5 mm, and 2.15 mm. The available smaller lumen tubes tended to have thicker sidewall thicknesses which adversely impacted the flexibility of the applicator tube making them unfit for use in MIS or robotic surgeries. Even the smaller applicator tubes that were tested had IDs which still permitted leakage of the powders and could be crushed by a robotic arm during use which would not allow for the flow of powder.

The solution needed to provide good flexibility in the applicator tube with little to no powder loss during movements of the handle and bellows in properly positioning the end of the applicator tube for application of the hemostatic powder. A number of solutions were attempted including varying the positions of the first crimp 42 and the second crimp, putting a flow restriction in the handle via tube insert or a molded component, and the like. The flow restrictor tube or molded component could also be placed in an adapter 16, to the bellows which would restrict the flow of powder.

The improvement defined by the present device provides inserting a small flow restriction tube member having a very small ID towards the handle, little to no powder spillage was observed without any sacrifice in the flexibility of the applicator tube and positionability of the powder delivery system.

As an illustrative example of a hemostatic powder dispenser assembly 2, the assembly includes a handle 12 that is between about 48 and 54 mm long, preferably about 50 to 52 mm long, and more preferably about 51.3 mm long. The proximal end of the flow restriction tube 44 is placed around 20 cm into the applicator tube 32 such that it covers the top half of the applicator tube/cannula 32. The first crimp is around 5 mm from the proximal end of the applicator tube and the second crimp is 56 mm from the first crimp. Thus, the second crimp 50 is about 61 mm from the proximal end of the applicator tube.

In another illustrative example of a hemostatic powder dispenser assembly 2, the flow restrictor tube 44 is no more than about 25% of the length of the applicator tube 32. In another example embodiment, the flow restrictor tube 44 is less than 50% of the length of the applicator tube 32.

Further testing was performed under ambient air conditions to compare this improvement to other suggested designs.

TABLE 1
Ambient Air Comparisons of Powder Loss.

| | 3 g powder loss | % compared to |
|---|---|---|
| PRODUCT | in 3 secs. | current product |
| On Market w/distal tip | 44% | N/A |
| On Market no tip | 35% | 21% less powder lost |
| Full Length 2.15 mm tube | 21% | 53% less powder lost |
| This improvement with | 6% | 86.6% less powder lost |
| 1.14 mm flow restrictor | | |

When the new and improved laparoscopic hemostatic powder device was tested in the lab under insufflated conditions and in in vivo (pig) testing there was almost no powder loss. The inventors conclude that under insufflated conditions the laparoscopic hemostatic powder device would not loose powder.

When the new and improved hemostatic powder dispenser assembly is used, the following protocol is applied:
1. Remove excess blood from the intended site by blotting, wiping or suctioning and identify and expose source of bleed;
2. Immediately apply a liberal amount of PerClot® ABSORBENT HEMOSTATIC POWDER to the source of bleeding by thoroughly covering the target bleed;
3. Apply direct pressure to the site using gauze for several minutes.
4. If bleeding continues, remove excess powder and repeat application.
5. Once hemostasis is achieved, remove excess powder carefully and completely by irrigation and aspiration.

The present device provides an improvement in a laparoscopic hemostatic powder delivery system which retains needed flexibility of the applicator tube while reducing or eliminating undesirable powder spillage and waste. This solution was accomplished with good manufacturing solutions that could be readily integrated into production with minimal additional costs. It was also unexpectedly discovered that creating a powder flow path with a series of constrictions and expansions repeated along the flow path made a turbulence which provided good puffing of the powder and excellent even spreading characteristics during application.

1. A hemostatic powder applicator comprising:
a hollow bellows container configured to store powder;
a handle member connected to the hollow bellows container, the handle member having an interior;
an applicator tube having a first length defining a flow path having a flow path diameter, the applicator tube having a proximal applicator end connected to the handle member and having a distal applicator end including a powder discharge opening; and
a flow restriction tube located within the flow path of the applicator tube, wherein the flow restriction tube includes a second length and a flow restriction tube inner diameter, wherein a flow restriction tube diameter is smaller than the flow path diameter and wherein the second length is shorter than the first length.

2. The hemostatic powder applicator according to claim 1, wherein the hemostatic powder applicator includes a first crimp and a second crimp, wherein the first crimp and the second crimp are configured to hold the flow restriction tube in place and to allow flow of powder from a distal container opening to the powder discharge opening.

3. The hemostatic powder applicator according to claim 2, wherein the inner diameter at the first crimp is larger than the inner diameter at the second crimp.

4. The hemostatic powder applicator according to claim 1, wherein the applicator tube and the flow restriction tube include an identical material.

5. The hemostatic powder applicator according to claim 1, wherein the flow restriction tube has a sidewall thickness of from about 2.5 to 3 mm and a length of from about 22.5 to 28 mm.

6. The hemostatic powder applicator according to claim 1, wherein the flow restriction tube has an outer diameter of from about 3 to 4 mm and an inner diameter of from about 1 to 1.5 mm, and wherein the applicator tube has an internal diameter of from about 3.5 to 4 mm and the outer diameter of from about 4.5 to 5.5 mm.

7. The hemostatic powder applicator according to claim 2, wherein the applicator tube and the flow restriction tube are configured to provide turbulence to hemostatic powder flowing through the inner diameter by providing larger inner diameter portions after and adjacent the distal tube end, the first crimp and the second crimp, respectively.

8. The hemostatic powder applicator according to claim 1, wherein the hemostatic powder includes PerClot® Absorbable Hemostatic Powder.

9. A hemostatic powder laparoscopic delivery system, including:
an applicator tube including a proximal applicator end and a distal applicator end, wherein the distal applicator end comprises a powder discharge opening;
a flow restriction tube positioned at the proximal applicator end, wherein the flow restriction tube comprises a proximal tube end and a distal tube end;
a handle member comprising a proximal handle end and a distal handle end, wherein the proximal handle end includes a proximal handle end opening and the distal handle end comprises a distal handle end opening; and
a hollow container comprising a proximal container end and a distal container end,
wherein hemostatic powder laparoscopic applicator comprises an inner diameter configured to allow a hemostatic powder to flow from a distal powder opening of the hollow container to the powder discharge opening.

10. The hemostatic powder laparoscopic delivery system according to claim 9, wherein the hemostatic powder laparoscopic delivery system includes
a first crimp configured to secure the proximal applicator end and the distal handle end, and
a second crimp configured to secure the distal tube end to the applicator tube,
wherein the first crimp and the second crimp are configured to cause the inner diameter to decrease from the distal container end of the hollow container to the powder discharge opening.

11. The hemostatic powder laparoscopic delivery system according to claim 9, wherein the proximal applicator end is telescopically received in the distal handle end opening.

12. The hemostatic powder laparoscopic delivery system according to claim 9, wherein the proximal handle end includes a threaded socket.

13. The hemostatic powder laparoscopic delivery system according to claim 9, wherein a threaded socket is configured to engage with a threaded distal portion of the hollow container.

14. The hemostatic powder laparoscopic delivery system according to claim 10, wherein the inner diameter at the first crimp is larger than the inner diameter at the second crimp.

15. The hemostatic powder laparoscopic delivery system according to claim 9, wherein the applicator tube and the flow restriction tube include an identical material.

16. The hemostatic powder laparoscopic delivery system according to claim 9, wherein the hemostatic powder is PerClot® Absorbable Hemostatic Powder.

17. The hemostatic powder laparoscopic delivery system according to claim 9, wherein the hemostatic powder laparoscopic delivery system further a grip secured to the handle member comprising two gripping handles.

18. The hemostatic powder laparoscopic delivery system according to claim 9, wherein the flow restriction tube includes an outer diameter of from about 3 to 4 mm and an inner diameter of from about 1 to 1.5 mm, and wherein the applicator tube has an internal diameter of from about 3.5 to 4 mm and an outer diameter of from about 4.5 to 5.5 mm.

19. A method of manufacturing a bellows type hemostatic powder dispenser, comprising a container, a handle member, and an applicator tube, wherein the method includes:
providing a flow restriction tube or similar dimensional restriction in the applicator or bellows;
inserting the flow restriction tube into the applicator tube to a specific depth within the applicator tube;
connecting the handle member to a side of the applicator tube as the flow restriction tube;
inserting a proximal end of a flow restrictor tube into a distal end of the handle member to a specified depth;
crimping the handle member, applicator tube and flow restrictor tube at a first location to provide a first crimp; and
crimping the applicator tube at a second location distal to the distal end of the flow restrictor tube to provide a second crimp.

20. The method according to claim 19, wherein the steps of crimping the handle member and crimping the applicator are performed at a same time.
